SEQUENCE LISTING (57 sequences)

SEQ ID NO: 1 - protein, 12 aa, Artificial Sequence
  Description of Artificial Sequence Formula peptide
WXXWXXEXXNYT

SEQ ID NO: 2 - protein, 68 aa, Artificial Sequence
  Description of Artificial Sequence Formula peptide
XXXXXXXXXXXXXXXXXXXXXXXXXXXXWXXWXXEXXNYTXXXXXXXXXXXXXXXXXXXXXXXXXXXX

SEQ ID NO: 3 - protein, 48 aa, Human immunodeficiency virus type 1
TTAVPWNASWSNKSLEDIWDNMTWMQWEREIDNYTNTIYTLLEESQNQ

SEQ ID NO: 4 - protein, 48 aa, Human immunodeficiency virus type 1
TTAVPWNASWSNKSLEQIWNNMTWMEWDREINNYTSLIHSLIEESQNQ

SEQ ID NO: 5 - protein, 48 aa, Human immunodeficiency virus type 1; identical to SEQ ID NO: 4

SEQ ID NO: 6 - protein, 48 aa, Human immunodeficiency virus type 1
TTAVPWNASWSNKSLSDIWDNMTWMEWEREIDNYTNLIYSLIEDSQIQ

SEQ ID NO: 7 - protein, 48 aa, Human immunodeficiency virus type 1; identical to SEQ ID NO: 4

SEQ ID NO: 8 - protein, 48 aa, Human immunodeficiency virus type 1
TTAVPWNASWSNKTLDQIWNNMTWMEWDREIDNYTHLIYTLIEESQNQ

SEQ ID NO: 9 - protein, 48 aa, Human immunodeficiency virus type 1
TTNVPWNSSWSNRSLNEIWQNMTWMEWEREIDNYTGLIYSLIEESQTQ

SEQ ID NO: 10 - protein, 48 aa, Human immunodeficiency virus type 1
TTAVPWNASWSNKSLEQIWNHTTWMEWDREINNYTSLIHSLIEESQNQ

SEQ ID NO: 11 - protein, 48 aa, Human immunodeficiency virus type 1; identical to SEQ ID NO: 10

SEQ ID NO: 12 - protein, 48 aa, Human immunodeficiency virus type 1
TTAVPWNASWSNKSLEEIWDNMTWMEWEREIDNYTSLIYTLIEESQNQ

SEQ ID NO: 13 - protein, 48 aa, Human immunodeficiency virus type 1
TTAVPWNTSWSNKSLDSIWNNMTWMEWEKEIENYTNTIYTLIEESQIQ

SEQ ID NO: 14 - protein, 48 aa, Human immunodeficiency virus type 1
TTAVPWNTSWSNKSFNEIWDNMTWMEWEREINNYTNLIYNLIEESQNQ

SEQ ID NO: 15 - protein, 48 aa, Human immunodeficiency virus type 1; identical to SEQ ID NO: 10

SEQ ID NO: 16 - protein, 48 aa, Human immunodeficiency virus type 1
TTFVPWNSSWSNRSLDDIWNNMTWMQWEKEISNYTGIIYNLIEESQIQ

SEQ ID NO: 17 - protein, 48 aa, Human immunodeficiency virus type 1
TTAVPWNASWSNKSLEQFWNNMTWMEWDREINNYTSLIHSLIDESQNQ

SEQ ID NO: 18 - protein, 48 aa, Human immunodeficiency virus type 1
TTTVPWNASWSNKSLDDIWNNMTWMQWEREIDNYTSLIYSLLEKSQTQ

SEQ ID NO: 19 - protein, 48 aa, Human immunodeficiency virus type 1
TTNVPWNSSWSNRSLDEIWQNMTWMEWEREIDNYTGLIYSLIEESQIQ

SEQ ID NO: 20 - protein, 48 aa, Human immunodeficiency virus type 1
TTTVPWNASWSNKSLNEIWDNMTWMQWEREIDNYTHLIYTLIEESQNQ

SEQ ID NO: 21 - protein, 48 aa, Human immunodeficiency virus type 1; identical to SEQ ID NO: 4

SEQ ID NO: 22 - protein, 48 aa, Human immunodeficiency virus type 1
TTTVPWNASWSNKSLNMIWNNMTWMQWEREIDNYTGIIYNLLEESQNQ

SEQ ID NO: 23 - protein, 48 aa, Human immunodeficiency virus type 1
TTAVPWNASWSNKSLDQIWNNMTWMEWEREIDNYTNLIYTLIEESQNQ

SEQ ID NO: 24 - protein, 48 aa, Human immunodeficiency virus type 1
TTTVPWNTSWSNKSLDKIWNNMTWMEWEREIDNYTSLIYTLLEESQNQ

SEQ ID NO: 25 - protein, 48 aa, Human immunodeficiency virus type 1
TTTVPWNTSWSNKSLDKIWGNMTWMEWEREIDNYTSLIYTLIEESQNQ

SEQ ID NO: 26 - protein, 48 aa, Human immunodeficiency virus type 1
TTTVPWNASWSNKSMDQIWNNMTWMEWEREIDNYTSLIYNLIEESQNQ

SEQ ID NO: 27 - protein, 48 aa, Human immunodeficiency virus type 1
TTTVPWNASWSNKSMNQIWDNLTWMEWEREIDNYTSIIYSLIEESQNQ

SEQ ID NO: 28 - protein, 48 aa, Human immunodeficiency virus type 1
TTTVPWNTSWSNKSLNEIWDNMTWMKWEREIDNYTHIIYSLIEQSQNQ

SEQ ID NO: 29 - protein, 48 aa, Human immunodeficiency virus type 1
TTTVPWNSSWSNRSLNDIWQNMTWMEWEREIDNYTGLIYRLIEESQTQ

SEQ ID NO: 30 - protein, 48 aa, Human immunodeficiency virus type 1; identical to SEQ ID NO: 29

SEQ ID NO: 31 - protein, 48 aa, Human immunodeficiency virus type 1
TTTVPWNSSWSNKSLEEIWNNMTWIEWEREIDNYTGVIYSLIENSQIQ

SEQ ID NO: 32 - protein, 45 aa, Human immunodeficiency virus type 1
PTNVPWNSSWSNKSQSDKMTWLEWDKEVSNYTQVIYNLIEESQTQ

SEQ ID NO: 33 - protein, 48 aa, Human immunodeficiency virus type 1
TTAVPWNASWSNKSLEQIWNNMTWMEWEREIDNYTSLIYSLIEESQNQ

SEQ ID NO: 34 - protein, 29 aa, Artificial Sequence
  Description of Artificial Sequence Synthetic peptide
SLEDIWDNMTWMQWEREIDNYTNTIYTLL

SEQ ID NO: 35 - protein, 29 aa, Artificial Sequence
  Description of Artificial Sequence Synthetic peptide
SLEQIWNNMTWMEWDREINNYTSLIHSLI

SEQ ID NO: 36 - protein, 29 aa, Artificial Sequence
  Description of Artificial Sequence Synthetic peptide
SLSDIWDNMTWMEWEREIDNYTNLIYSLI

SEQ ID NO: 37 - protein, 29 aa, Artificial Sequence
  Description of Artificial Sequence Synthetic peptide
TLDQIWNNMTWMEWDREIDNYTHLIYTLI

SEQ ID NO: 38 - protein, 29 aa, Artificial Sequence
  Description of Artificial Sequence Synthetic peptide
SLNEIWQNMTWMEWEREIDNYTGLIYSLI

SEQ ID NO: 39 - protein, 29 aa, Artificial Sequence
  Description of Artificial Sequence Synthetic peptide
SLEQIWNHTTWMEWDREINNYTSLIHSLI

SEQ ID NO: 40 - protein, 29 aa, Artificial Sequence
  Description of Artificial Sequence Synthetic peptide
SLEEIWDNMTWMEWEREIDNYTSLIYTLI

SEQ ID NO: 41 - protein, 29 aa, Artificial Sequence
  Description of Artificial Sequence Synthetic peptide
SLDSIWNNMTWMEWEKEIENYTNTIYTLI

SEQ ID NO: 42 - protein, 29 aa, Artificial Sequence
  Description of Artificial Sequence Synthetic peptide
SFNEIWDNMTWMEWEREINNYTNLIYNLI

SEQ ID NO: 43 - protein, 29 aa, Artificial Sequence
  Description of Artificial Sequence Synthetic peptide
SLDDIWNNMTWMQWEKEISNYTGIIYNLI

SEQ ID NO: 44 - protein, 29 aa, Artificial Sequence
  Description of Artificial Sequence Synthetic peptide
SLEQFWNNMTWMEWDREINNYTSLIHSLI

SEQ ID NO: 45 - protein, 29 aa, Artificial Sequence
  Description of Artificial Sequence Synthetic peptide
SLDDIWNNMTWMQWEREIDNYTSLIYSLL

SEQ ID NO: 46 - protein, 29 aa, Artificial Sequence
  Description of Artificial Sequence Synthetic peptide
SLDEIWQNMTWMEWEREIDNYTGLIYSLI

SEQ ID NO: 47 - protein, 29 aa, Artificial Sequence
  Description of Artificial Sequence Synthetic peptide
SLNEIWDNMTWMQWEREIDNYTHLIYTLI

SEQ ID NO: 48 - protein, 29 aa, Artificial Sequence
  Description of Artificial Sequence Synthetic peptide
SLNMIWNNMTWMQWEREIDNYTGIIYNLL

SEQ ID NO: 49 - protein, 29 aa, Artificial Sequence
  Description of Artificial Sequence Synthetic peptide
SLDQIWNNMTWMEWEREIDNYTNLIYTLI

SEQ ID NO: 50 - protein, 29 aa, Artificial Sequence
  Description of Artificial Sequence Synthetic peptide
SLDKIWNNMTWMEWEREIDNYTSLIYTLL

SEQ ID NO: 51 - protein, 29 aa, Artificial Sequence
  Description of Artificial Sequence Synthetic peptide
SLDKIWGNMTWMEWEREIDNYTSLIYTLI

SEQ ID NO: 52 - protein, 29 aa, Artificial Sequence
  Description of Artificial Sequence Synthetic peptide
SMDQIWNNMTWMEWEREIDNYTSLIYNLI

SEQ ID NO: 53 - protein, 29 aa, Artificial Sequence
  Description of Artificial Sequence Synthetic peptide
SMNQIWDNLTWMEWEREIDNYTSIIYSLI

SEQ ID NO: 54 - protein, 29 aa, Artificial Sequence
  Description of Artificial Sequence Synthetic peptide
SLNEIWDNMTWMKWEREIDNYTHIIYSLI

SEQ ID NO: 55 - protein, 29 aa, Artificial Sequence
  Description of Artificial Sequence Synthetic peptide
SLNDIWQNMTWMEWEREIDNYTGLIYRLI

SEQ ID NO: 56 - protein, 29 aa, Artificial Sequence
  Description of Artificial Sequence Synthetic peptide
SLEEIWNNMTWIEWEREIDNYTGVIYSLI

SEQ ID NO: 57 - protein, 29 aa, Artificial Sequence
  Description of Artificial Sequence Synthetic peptide
SQSDIWDKMTWLEWDKEVSNYTQVIYNLI

(19) United States
(12) Patent Application Publication
(10) Pub. No.: US 2004/0002581 A1
(43) Pub. Date: Jan. 1, 2004

(54) Peptides and use thereof in therapeutic agents against HIV infection

(75) Inventor: Pierre F. Serres, Saint-Genis Laval (FR)

Correspondence Address: PILLSBURY WINTHROP, LLP, P.O. BOX 10500, MCLEAN, VA, 22102, US

(73) Assignee: Mymetics Corp., Annapolis, MD

(21) Appl. No.: 10/305,133

(22) Filed: Nov. 27, 2002

Related U.S. Application Data
(60) Provisional application No. 60/340,492, filed on Dec. 18, 2001.

(30) Foreign Application Priority Data
Nov. 29, 2001 (FR) 0115423

(51) Int. Cl. A61K 38/16; C07K 14/00

(52) U.S. Cl. 530/324; 514/12

(57) ABSTRACT

The present invention provides novel peptides A peptide containing from 25 to 40 amino acids and comprising a sequence π corresponding to formula I;

(WX₁X₂WX₃X₄EX₅X₆NYT) (SEQ ID NO: 1), wherein X1 represents an amino acid selected from the group consisting of amino acids M, I, L and Q; X₂ represents an amino acid selected from the group consisting of amino acids E, K, R, and Q; X₃ represents an amino acid selected from the group consisting of amino acids D and E; X₄ represents an amino acid selected from the group consisting of K, R, and Q; X₅ represents and amino acid selected from the group consisting of amino acids V, I, and L; X₆ represents and amino acid selected from the group consisting of amino acids D, S, N, and E; and wherein letters in formula I other than X represent amino acids according to conventional nomenclature.

### RELATED APPLICATIONS

This application claims priority to French Application No. FR 0115423 filed Nov. 29, 2001 and U.S. Provisional Application No. 60/340,492, filed Dec. 18, 2001. The contents of both applications are herein incorporate by reference in their entirety.

### FIELD OF THE INVENTION

The present invention relates the identification and design of new peptides and their use as therapeutic agents against human infection with the human immunodeficiency virus (HIV).

### DESCRIPTION OF RELATED ART

Since the recognition of human acquired immunodeficiency syndrome (AIDS) in 1981, there has been intensive research into the causal virus, human immunodeficiency virus (HIV), formerly known as human T-cell lymphotropic virus type III (HTLV-III) or lymphadenopathy-associated virus (LAV). It is now known that HIV-specific antibodies are present in the sera not only of most patients with AIDS or AIDS-related complex, but also in the sera of asymptomatic individuals exposed to the virus.

More recently, a variant virus, known as HIV-2, has also been found to be capable of causing AIDS. Immunoassay methods, such as ELISA, utilizing various polypeptides encoded by the HIV virus have been extensively used in diagnosis and screening. In most cases the polypeptides are either directly prepared from viral material, or are derived from in vitro expression systems using recombinant DNA technology, although such materials are not ideal. Material derived from viral preparations may be contaminated by viable virus, thus posing a hazard to personnel using the material. Recombinant-derived material may be contaminated by non-HIV protein, resulting in possible loss of specificity.

Human immunodeficiency virus, like all other viruses, is necessary parasite which in order to multiply must penetrate into the cell of a host, particularly, a T lymphocyte of a host.

It is known that in the treatment of AIDS in humans existing therapies use the so called “triple therapy” regiment which is costly and now is more and more questionable due to the emergence of HIV virus strains that are resistant to the drugs that make up the “triple therapy” regimen.

Based on the high cost of therapy regimens such as the “triple therapy” regimen, it is not conceivable that such treatments would be made available for the treatment of domestic cats infected with the FIV virus.

By contrast, peptide based therapies present a cost effective alternative to the drugs used in the triple therapy regimen.

In recent years, peptides have been developed as inhibitors against HIV infection of host cells. In particular, peptides DP 107, DP 219 and DP 178 corresponding to the fragments 558-595, 635-664 and 643-678 of the HIV transmembrane envelope protein gp41 with a numbering corresponding to the numbering used in Tables 1 and 2 provided below have been investigated as inhibitors of virus entry into target cells. Developed.

The mechanism of action of the peptides investigated to date in connection with HIV infection inhibition has not been elucidated. However, it is generally accepted that these peptides act by block entry of the virus into the target cells.

While the peptides known to date have shown very promising results in blocking HIV infection. There remains a need for more peptide candidates that may provide efficacious medicine for peptide based effective therapeutic agents.

### SUMMARY OF THE INVENTION

The present invention provides novel peptides A peptide containing from 25 to 40 amino acids and comprising a sequence π corresponding to formula I:

WX₁X₂WX₃X₄EX₅X₆NYT

wherein X1 represents an amino acid selected from the group consisting of amino acids M, I, L and Q; X₂ represents an amino acid selected from the group consisting of amino acids E, K, R, and Q; X₃ represents an amino acid selected from the group consisting of amino acids D and E; X₄ represents an amino acid selected from the group consisting of amino acids K, R, and Q; X₅ represents an amino acid selected from the group consisting of amino acids V, I, and L; and X₆ represents an amino acid selected from the group consisting of amino acids D, S, N, and E; and wherein letters in formula I other than X represent amino acids according to conventional nomenclature.

In a preferred embodiment, the invention provides peptide corresponding to formula II:

(X₇)ₘ-π-(X8)ₙ

wherein π is as defined above; X₇ represents a peptides containing at most 28 amino acids; X₈ represents a peptides containing at most 28 amino acids;

or X₇ and X₈ together represent a divalent peptide which forms with π a cyclic peptide; m is 0 or 1, n is 0 or 1; with the proviso that n and m cannot at the same time be equal to 0. Optionally, X7, X8 or both correspond to an amino acid sequence such that the peptide of formula II has a secondary structure corresponding to an α helix.

X7 corresponds to the formula:

(X₇″)ₘ-X₇′-

wherein X7′ represents amino acid T₆₃₂ of a gp41 protein from an HIV-1 strain or a fragment contained in a sequence corresponding to amino acids 610 to 632 of a gp41 protein from an HIV-1 strain, a consensus sequence corresponding thereto, or a sequence obtained by replacing in a fragment contained in a sequence corresponding to amino acids 610 to 632 of a gp41 protein from an HIV-1 strain or a consensus sequence corresponding thereto K with R, R with K, I with L, L with I or K and R with Q, wherein X7′ is linked to [symbol] through amino acid 632, and wherein X7″ represents one amino acid or a fragment of a peptide containing at most 27 amino acids; and m′ is 0 or 1.

Preferred peptides are those wherein the amino acids forming the fragment contained in a sequence corresponding to amino acids 610 to 632 of a gp41 protein from an HIV-1 strain are selected from corresponding amino acids in the sequences of Tables 1 and 2.

In a another preferred embodiment, the invention provides peptide wherein X8 corresponds to the formula:

-X₈′-(X₈″)_{n′}

wherein X8′ represents N₆₄₅, S₆₄₅, G₆₄₅, H₆₄₅ or Q645 of a gp41 protein from an HIV-1 strain or a fragment contained in a sequence corresponding to amino acids 657 to 657 of a gp41 protein from an HIV-1 strain, a consensus sequence corresponding thereto, or a sequence obtained by replacing in a fragment contained in a sequence corresponding to amino acids 645 to 657 of a gp41 protein from an HIV-1 strain or a consensus sequence corresponding thereto K with R, R with K, I with L, L with I or K and R with Q, wherein X8′ is linked to [symbol] through amino acid 645, X8″ represents one amino acid or a fragment of a peptide containing at most 27 amino acids; and n′ is 0 or 1. Preferably, the amino acids forming the fragment contained in a sequence corresponding to amino acids 645 to 657 of a gp41 protein from an HIV-1 strain are selected from corresponding amino acids in the sequences of Tables 1 and 2.

The invention also provides therapeutic agents comprising one or more peptides according to the invention in combination with an excipient and/or a pharmaceutical carrier.

The invention also provides a method of treating a subject infected with HIV virus, wherein the method comprises administering to the subject a therapeutically effective amount of a therapeutic agent comprising one or more peptides according to the invention.

The invention further provides a method of screening for HIV infection comprising:

a) attaching a peptide according to the invention to a solid support,

b) contacting a biological sample with the peptide to allow binding of anti-HIV antibodies to the peptide for a time sufficient to form a peptide-antibody complex,

c) reacting said complex with anti-HIV antibodies, and

d) detecting the anti-HIV antibodies if present.

Preferred antibodies have a label selected from the group consisting of a radioisotope, an enzyme, and a fluorophore.

### DETAILED DESCRIPTION OF SELECTED EMBODIMENTS OF THE INVENTION

As indicated above, the present invention provides novel peptides containing from 25 to 40 amino acids and comprising a sequence π corresponding to formula I:

WX₁X₂WX₃X₄EX₅X₆NYT

wherein X1 represents an amino acid selected from the group consisting of amino acids M, I, L and Q; X₂ represents an amino acid selected from the group consisting of amino acids E, K, R, and Q; X₃ represents an amino acid selected from the group consisting of amino acids D and E; X₄ represents an amino acid selected from the group consisting of amino acids K, R, and Q; X₅ represents an amino acid selected from the group consisting of amino acids V, I, and L; and X₆ represents an amino acid selected from the group consisting of amino acids D, S, N, and E; and wherein letters in formula I other than X represent amino acids according to conventional nomenclature.

The peptides of the invention are advantageous in that they provide an avenue for developing effective therapies against HIV infection at a cost lower than the cost of the existing therapies. The peptides of the invention are designed based on a highly conserved region of the envelope protein gp41 of the HIV virus which in turn makes therapies based on the peptides of the invention useful against most strains of the virus. In addition, the degree of conservation associated with the region of the gp41 protein used as a basis for the design of the subject peptides reduces significantly the ability of the virus to undergo mutations that would diminish the efficacy of therapies based on the peptides of the invention.

In a preferred embodiment, the invention provides peptide corresponding to formula II:

(X₇)ₘ-π-(X₈)ₙ

wherein π is as defined above; X₇ represents a peptides containing at most 28 amino acids; X₈ represents a peptides containing at most 28 amino acids;

or X₇ and X₈ together represent a divalent peptide which forms with π a cyclic peptide; m is 0 or 1, n is 0 or 1; with the proviso that n and m cannot at the same time be equal to 0. Optionally, X7, X8 or both correspond to an amino acid sequence such that the peptide of formula II has a secondary structure corresponding to an α helix.

X7 and X8 may represent a modified or unmodified natural amino acid selected from the known 20 amino acids or a non natural (synthetic) amino acid. Examples of modified natural amino acids N-acetylated peptides, particularly for peptides positioned at the N terminal of the polypeptide, and hydroxylated proline amino acids. The presence of modified natural amino acids in the peptides of the invention generally confers more stability to the peptides, particularly against possible degradation by enzymes. Examples of non natural amino acids that may be used in the design of the peptides of the invention include α-amino isobutyric acid.

The peptides of the invention may be in cyclical form. Cyclic peptides according to the invention may be obtained, for example, by positioning in X7 and/or X8 at least two cysteine residues with the formation of a disulfide bond between the two cysteine residues or the presence of a lysine residue and an asparatic acid residue that form a lactame bridge.

In one preferred embodiment, the sequences of X7 and X8 are selected such that the peptide of formula II has a secondary structure containing an a helix. The presence of an a helix in a peptide according to the invention can be shown through conventional methods such as circular dichroism.

Thus, X7 and/or X8 may contain a relatively high proportion of amino acids that have a propensity to form a helices. Examples of amino acids known to favor a helix formation include leucine, lysine and glutamic acid. X7 and/or X8 may also contain one or more residues corresponding to α-amino isobutyric acid, which is known to favor a helix formation (see for example J. Venkatraman et al., Chem. Rev. 2001, 101, 3131, 3152.

X7 and X8 may be chosen from any peptide or protein fragment known to adopt an α helix structure. Examples of such fragments include fragment extracted from the sequence of myoglobin.

As well, X7 and X8 can also contain fragments from the sequence of the envelope protein gp41.

In a preferred embodiment X7 corresponds to the formula:

(X₇″)_{m′}-X₇′-

wherein X7′ represents amino acid T₆₃₂ of a gp41 protein from an HIV-1 strain or a fragment contained in a sequence corresponding to amino acids 610 to 632 of a gp41 protein from an HIV-1 strain, a consensus sequence corresponding thereto, or a sequence obtained by replacing in a fragment contained in a sequence corresponding to amino acids 610 to 632 of a gp41 protein from an HIV-1 strain or a consensus sequence corresponding thereto K with R, R with K, I with L, L with I or K and R with Q, wherein X7′ is linked to [symbol] through amino acid 632, and wherein X7″ represents one amino acid or a fragment of a peptide containing at most 27 amino acids; and m′ is 0 or 1.

When X7′ represents a peptide contained in the sequence 610-632 of the gp41 protein and linked to n by amino acid 632, X7′ represents a peptide fragment corresponding to the entire C terminal of the sequence 610-632 of gp41 or a fragment thereof consistent while preserving the continuity of the sequence.

Preferred peptides are those wherein the amino acids forming the fragment contained in a sequence corresponding to amino acids 610 to 632 of a gp41 protein from an HIV-1 strain are selected from corresponding amino acids in the sequences of Tables 1 and 2.

In a another preferred embodiment, the invention provides peptide wherein X8 corresponds to the formula:

-X₈′-(X₈″)_{n′}

wherein X8′ represents N₆₄₅, S₆₄₅, G₆₄₅, H₆₄₅ or Q₆₄₅ of a gp41 protein from an HIV-1 strain or a fragment contained in a sequence corresponding to amino acids 657 to 657 of a gp41 protein from an HIV-1 strain, a consensus sequence corresponding thereto, or a sequence obtained by replacing in a fragment contained in a sequence corresponding to amino acids 645 to 657 of a gp41 protein from an HIV-1 strain or a consensus sequence corresponding thereto K with R, R with K, I with L, L with I or K and R with Q, wherein X8′ is linked to [symbol] through amino acid 645, X8″ represents one amino acid or a fragment of a peptide containing at most 27 amino acids; and n′ is 0 or 1. Preferably, the amino acids forming the fragment contained in a sequence corresponding to amino acids 645 to 657 of a gp41 protein from an HIV-1 strain are selected from corresponding amino acids in the sequences of Tables 1 and 2.

When X8′ represents a peptide contained in the sequence 645-657 of the gp41 protein and linked to [symbol] by amino acid 645, X8′ represents a peptide fragment corresponding to the entire C terminal of the sequence 645-657 of gp41 or a fragment thereof consistent while preserving the continuity of the sequence.

The numbering used herein is consistent with conventional numbering wherein peptides and peptide sequences are transcribed from the N terminal amino acid to the C terminal amino acid. Accordingly, for all the peptides and peptide sequences presented herein the left extremity corresponds the N terminal amino acid and the right extremity corresponds to the C terminal amino acid.

Table 1, appended below, contains sequences corresponding to the positions 610-657 of the gp41 envelope protein extracted from various HIV strains. The sequences provided in Table 1 allow easy identification of the amino acids to be selected in the design of peptides according to the invention as described above. It should be noted that the invention also encompasses sequences obtained by replacing in a sequence listed in Table 1 K with R, R with K, I with L, L with I or K and R with Q. Table 2, also appended below, shows a consensus sequence for the fragment 610-657 of the gp41 protein.

While the invention has been illustrated through sequences designed based on the fragment 610-657 of the protein gp41, it is expected that the invention can be carried out using a sequence of a gp41 molecule corresponding to any known HIV strain or HIV strain to be uncovered and/or published.

The invention also provides therapeutic agents comprising one or more peptides according to the invention in combination with an excipient and/or a pharmaceutical carrier.

The invention also provides a method of treating a subject infected with HIV virus, wherein the method comprises administering to the subject a therapeutically effective amount of a therapeutic agent comprising one or more peptides according to the invention. The peptides can be administered in a dosage ranging from 0.1 to 2 mg/kg of body weight.

A therapeutic agent according to the invention will contain at least one peptide as defined herein, and possibly an excipient and/or a suitable pharmaceutical carrier. The therapeutic agent can be administered, for example, parenterally or through an IV injection. The therapeutic agent may be in the form of a solution or injectable suspension or in the form of lyophilized powder containing the peptide as active agent, with optionally a conventional lyophilization adjuvant. The lyophilized powder allows the reconstitution of the injectable suspension.

The invention further provides a method of screening for HIV infection comprising:

a) attaching a peptide according to the invention to a solid support,

b) contacting a biological sample with the peptide to allow binding of anti-HIV antibodies to the peptide for a time sufficient to form a peptide-antibody complex,

c) reacting said complex with anti-HIV antibodies, and

d) detecting the anti-HIV antibodies if present.

Preferred antibodies have a label selected from the group consisting of a radioisotope, an enzyme, and a fluorophore.

Effectiveness of the peptides of the invention in inhibiting infection by the HIV virus can be shown through numerous well known tests and techniques. In particular, infection inhibition can be shown through the inhibition of syncitia formation. Another way of showing infection inhibition is based on an ELISA test showing a decrease in the production of the antigen p24 (see for example Jian et al. J. Exp. Med. 174:1557-63 (1991)). In the latter test, peptides according to the invention decrease the production of the antigen p24 and a dose dependent manner.

Preferred peptides according to the present invention are peptides that significantly inhibit HIV infection by allowing a decrease in viral infection by at least 20%, and more preferably 50%, measured by dosing the antigen p24. An example of a protocol for measuring the impact of a peptide of the invention on the production of the antigen p24 is provided below.

### EXAMPLES

The following Example illustrates the invention.

### Example 1

A peptide having the sequence corresponding amino acids 623-651 presented in Table 2 was synthesized. Starting with a solution containing 50 μg of the synthesized peptide, a series of dilutions were carried out in RPMI 1640. A 50 μl sample is disposed in a well of a 96 well array. Then 25 μl of suspension containing the HIV-1 virus are disposed in each well with a viral concentration of 100 times cell ID₅₀ (dose for 50% infection). Human cells MT-2 are then added at a concentration of 0.5×10⁶ cells/ml are then added to each well sample. Incubation is allowed for six days after which the supernatant is collected and tested to determine the concentration of the antigen p24 according to the ELISA test described below.

Titration of the antigen p24 with the ELISA method: Dosages of 100 μl of a 10 μg/ml preparation containing sheep anti-p24 antibodies purified by affinity column and commercialized under the trade name D7320 by Aalto, and NaHCO₃ buffer (100 mM, pH 8.5) are disposed in the wells of a F16 Maxisorb plate and incubated overnight. After washing with a saline buffered solution (NaCl 1.44M, Tween 20 0.5%, Tris 250 mM, pH 7.5) the blocking is carried out during 30 minutes with a TBS buffer containing 2% powder milk.

After washing, 100 μl of various dilutions obtained based on a solution of antigen p24 obtained from the isolate LAI/BRU (Medical Research Council AIDS Reagent Project MRC ARP), are added to the wells and incubation is carried out overnight at room temperature. Next, a preparation of biotinylated murine anti-p24 monoclonal antibodies (100 μl, MRC ARP) obtained by a 1,000 dilution is blocked for two hours in a TMT/SS buffer (blocking buffer containing 20% sheep serum and 0.5% Tween 20). After six washings with the TBS buffer, 100 ml of the conjugate alkaline streptavidine phosphatase (1:500 in TMT/SS, Boehringer Mannheim) are added. The mixture is then incubated for one hour.

After six washings with the TBS buffer, a p-nitrophenylphosphate solution (10 mg for 10 ml; ethanolamine 10 mM, MgCl₂ 0.5 mM, pH 9.5, Sigma) is added. Absorption in the wave length rang 405-690 nm is then detected.

The experiment showed that the peptide corresponding to the sequence 623-651 of Table 2 markedly reduces the quantity of p24 antigen resent in the supernatant and significantly inhibits HIV infection.

### Example 2

Peptides corresponding to the sequences listed in Table 3 are synthesized and tested according to the protocol described in Example 1.

TABLE 1

| 1. ENV_HV1A2 | | T | T | A | V | P | W | N | A | S | W | S | N | K | S | L | E |
|---|---|---|---|---|---|---|---|---|---|---|---|---|---|---|---|---|---|
| 2. ENV_HV1B1 | | T | T | A | V | P | W | N | A | S | W | S | N | K | S | L | E |
| 3. ENV_HIV1B8 | | T | T | A | V | P | W | N | A | S | W | S | N | K | S | L | E |
| 4. ENV_HIV1BN | | T | T | A | V | P | W | N | A | S | W | S | N | K | S | L | S |
| 5. ENV_HIV1BR | | T | T | A | V | P | W | N | A | S | W | S | N | K | S | L | E |
| 6. ENV_HIV1C4 | | T | T | A | V | P | W | N | A | S | W | S | N | K | T | L | D |
| 7. ENV_HIV1EL | | T | T | N | V | P | W | N | S | S | W | S | N | R | S | L | N |
| 8. ENV_HIV1H2 | | T | T | A | V | P | W | N | A | S | W | S | N | K | S | L | E |
| 9. ENV_HIV1H3 | | T | T | A | V | P | W | N | A | S | W | S | N | K | S | L | E |
| 10. ENV_HIV1J3 | | T | T | A | V | P | W | N | A | S | W | S | N | K | S | L | E |
| 11. ENV_HIV1JR | | T | T | A | V | P | W | N | T | S | W | S | N | K | S | L | D |
| 12. ENV_HIV1KB | | T | T | A | V | P | W | N | T | S | W | S | N | K | S | F | N |
| 13. ENV_HIV1LW | | T | T | A | V | P | W | N | A | S | W | S | N | K | S | L | E |
| 14. ENV_HIV1MA | | T | T | F | V | P | W | N | S | S | W | S | N | R | S | L | D |
| 15. ENV_HIV1MF | | T | T | A | V | P | W | N | A | S | W | S | N | K | S | L | E |
| 16. ENV_HIV1MN | | T | T | T | V | P | W | N | A | S | W | S | N | K | S | L | D |
| 17. ENV_HIV1ND | | T | T | N | V | P | W | N | S | S | W | S | N | R | S | L | D |
| 18. ENV_HIV1OY | | T | T | T | V | P | W | N | A | S | W | S | N | K | S | L | N |
| 19. ENV_HIV1PV | | T | T | A | V | P | W | N | A | S | W | S | N | K | S | L | E |
| 20. ENV_HIV1RH | | T | T | T | V | P | W | N | A | S | W | S | N | K | S | L | N |
| 21. ENV_HIV1S1 | | T | T | A | V | P | W | N | A | S | W | S | N | K | S | L | D |
| 22. ENV_HIV1S3 | | T | T | T | V | P | W | N | T | S | W | S | N | K | S | L | D |
| 23. ENV_HIV1SC | | T | T | T | V | P | W | N | T | S | W | S | N | K | S | L | D |
| 24. ENV_HIV1W1 | | T | T | T | V | P | W | N | A | S | W | S | N | K | S | M | D |
| 25. ENV_HIV1W2 | | T | T | T | V | P | W | N | A | S | W | S | N | K | S | M | N |
| 26. ENV_HIV1Y2 | | T | T | T | V | P | W | N | T | S | W | S | N | K | S | L | N |
| 27. ENV_HIV1Z2 | | T | T | T | V | P | W | N | S | S | W | S | N | R | S | L | N |
| 28. ENV_HIVIZ6 | | T | T | T | V | P | W | N | S | S | W | S | N | R | S | L | N |
| 29. ENV_HIVIZ8 | | T | T | T | V | P | W | N | S | S | W | S | N | K | S | L | E |
| 30. ENV_HIV1ZH | | P | T | N | V | P | W | N | S | S | W | S | N | K | S | Q | S |
| Numèrotation | AA | 610 | 611 | 612 | 613 | 614 | 615 | 616 | 617 | 618 | 619 | 620 | 621 | 622 | 623 | 624 | 625 |
| 1. ENV_HV1A2 | | D | I | W | D | N | M | T | W | M | Q | W | E | R | E | I | D |
| 2. ENV_HV1B1 | | Q | I | W | N | N | M | T | W | M | E | W | D | R | E | I | N |
| 3. ENV_HIV1B8 | | Q | I | W | N | N | M | T | W | M | E | W | D | R | E | I | N |
| 4. ENV_HIV1BN | | D | I | W | D | N | M | T | W | M | E | W | E | R | E | I | D |
| 5. ENV_HIV1BR | | Q | I | W | N | N | M | T | W | M | E | W | D | R | E | I | N |
| 6. ENV_HIV1C4 | | Q | I | W | N | N | M | T | W | M | E | W | D | R | E | I | D |
| 7. ENV_HIV1EL | | E | I | W | Q | N | M | T | W | M | E | W | E | R | E | I | D |
| 8. ENV_HIV1H2 | | Q | I | W | N | H | T | T | W | M | E | W | D | R | E | I | N |
| 9. ENV_HIVIH3 | | Q | I | W | N | H | T | T | W | M | E | W | D | R | E | I | N |
| 10. ENV_HIV1J3 | | E | I | W | D | N | M | T | W | M | E | W | E | R | E | I | D |
| 11. ENV_HIV1JR | | S | I | W | N | N | M | T | W | M | E | W | E | K | E | I | E |
| 12. ENV_HIV1KB | | E | I | W | D | N | M | T | W | M | E | W | E | R | E | I | N |
| 13. ENV_HIVILW | | Q | I | W | N | H | T | T | W | M | E | W | D | R | E | I | N |
| 14. ENV_H1V1MA | | D | I | W | N | N | M | T | W | M | Q | W | E | K | E | I | S |
| 15. ENV_HIV1MF | | Q | F | W | N | N | M | T | W | M | E | W | D | R | E | I | N |
| 16. ENV_HIV1MN | | D | I | W | N | N | M | T | W | M | Q | W | E | R | E | I | D |
| 17. ENV_HIV1ND | | E | I | W | Q | N | M | T | W | M | E | W | E | R | E | I | D |
| 18. ENV_HIV1OY | | E | I | W | D | N | M | T | W | M | Q | W | E | R | E | I | D |
| 19. ENV_HIV1PV | | Q | I | W | N | N | M | T | W | M | E | W | D | R | E | I | N |
| 20. ENV_HIV1RH | | M | I | W | N | N | M | T | W | M | Q | W | E | R | E | I | D |
| 21. ENV_HIV1S1 | | Q | I | W | N | N | M | T | W | M | E | W | E | R | E | I | D |
| 22. ENV_HIV1S3 | | K | I | W | N | N | M | T | W | M | E | W | E | R | E | I | D |
| 23. ENV_HIV1SC | | K | I | W | G | N | M | T | W | M | E | W | E | R | E | I | D |
| 24. ENV_HIV1W1 | | Q | I | W | N | N | M | T | W | M | E | W | E | R | E | I | D |
| 25. ENV_H1V1W2 | | Q | I | W | D | N | L | T | W | M | E | W | E | R | E | I | D |
| 26. ENV_HIV1Y2 | | E | I | W | D | N | M | T | W | M | K | W | E | R | E | I | D |
| 27. ENV_HIV1Z2 | | D | I | W | Q | N | M | T | W | M | E | W | E | R | E | I | D |
| 28. ENV_HIVIZ6 | | D | I | W | Q | N | M | T | W | M | E | W | E | R | E | I | D |
| 29. ENV_HIV1Z8 | | E | I | W | N | N | M | T | W | I | E | W | E | R | E | I | D |
| 30. ENV_HIV1ZH | | D | I | W | D | K | M | T | W | L | E | W | D | K | E | V | S |
| Numèrotation | AA | 626 | 627 | 628 | 629 | 630 | 631 | 632 | 633 | 634 | 635 | 636 | 637 | 638 | 639 | 640 | 641 |
| 1. ENV_HV1A2 | | N | Y | T | N | T | I | Y | T | L | L | E | E | S | Q | N | Q |
| 2. ENV_HV1B1 | | N | Y | T | S | L | I | H | S | L | I | E | E | S | Q | N | Q |
| 3. ENV_HIV1B8 | | N | Y | T | S | L | I | H | S | L | I | E | E | S | Q | N | Q |
| 4. ENV_HIV1BN | | N | Y | T | N | L | I | Y | S | L | I | E | D | S | Q | I | Q |
| 5. ENV_H1V1BR | | N | Y | T | S | L | I | H | S | L | I | E | E | S | Q | N | Q |
| 6. ENV_HIV1C4 | | N | Y | T | H | L | I | Y | T | L | I | E | E | S | Q | N | Q |
| 7. ENV_HIV1EL | | N | Y | T | G | L | I | Y | S | L | I | E | E | S | Q | T | Q |
| 8. ENV_H1V1H2 | | N | Y | T | S | L | I | H | S | L | I | E | E | S | Q | N | Q |
| 9. ENV_HIV1H3 | | N | Y | T | S | L | I | H | S | L | I | E | E | S | Q | N | Q |
| 10. ENV_HIV1J3 | | N | Y | T | S | L | I | Y | T | L | I | E | E | S | Q | N | Q |
| 11. ENV_HJV1R | | N | Y | T | N | T | I | Y | T | L | I | E | E | S | Q | I | Q |
| 12. ENV_HIV1KB | | N | Y | T | N | L | I | Y | N | L | I | E | E | S | Q | N | Q |
| 13. ENV_HIV1LW | | N | Y | T | S | L | I | H | S | L | I | E | E | S | Q | N | Q |
| 14. ENV_HIV1MA | | N | Y | T | G | I | I | Y | N | L | I | E | E | S | Q | I | Q |
| 15. ENV_HIV1MF | | N | Y | T | S | L | I | H | S | L | I | D | E | S | Q | N | Q |
| 16. ENV_HIV1MN | | N | Y | T | S | L | I | Y | S | L | L | E | K | S | Q | T | Q |
| 17. ENV_HIV1ND | | N | Y | T | G | L | I | Y | S | L | I | E | E | S | Q | I | Q |
| 18. ENV_HIV1OY | | N | Y | T | H | L | I | Y | T | L | I | E | E | S | Q | N | Q |
| 19. ENV_HIV1PV | | N | Y | T | S | L | I | H | S | L | I | E | E | S | Q | N | Q |
| 20. ENV_H1V1RH | | N | Y | T | G | 1 | I | Y | N | L | L | E | E | S | Q | N | Q |
| 21. ENV_HIV1S1 | | N | Y | T | N | L | I | Y | T | L | I | E | E | S | Q | N | Q |
| 22. ENV_HIV1S3 | | N | Y | T | S | L | I | Y | T | L | L | E | E | S | Q | N | Q |
| 23. ENV_HIV1SC | | N | Y | T | S | L | I | Y | T | L | I | E | E | S | Q | N | Q |
| 24. ENV_HIV1W1 | | N | Y | T | S | L | I | Y | N | L | I | E | E | S | Q | N | Q |
| 25. ENV_HIV1W2 | | N | Y | T | S | I | I | Y | S | L | I | E | E | S | Q | N | Q |
| 26. ENV_HIV1Y2 | | N | Y | T | H | I | I | Y | S | L | I | E | Q | S | Q | N | Q |
| 27. ENV_HIV1Z2 | | N | Y | T | G | L | I | Y | R | L | I | E | E | S | Q | T | Q |
| 28. ENV_HIV1Z6 | | N | Y | T | G | L | I | Y | R | L | I | E | E | S | Q | T | Q |
| 29. ENV_HIV1Z8 | | N | Y | T | G | V | I | Y | S | L | I | E | N | S | Q | I | Q |
| 30. ENV_HIV1ZH | | N | Y | T | Q | V | I | Y | N | L | I | E | E | S | Q | T | Q |
| Numerotation | AA | 642 | 643 | 644 | 645 | 646 | 647 | 648 | 649 | 650 | 651 | 652 | 653 | 654 | 655 | 656 | 657 |

TABLE 2

| | T | T | A | V | P | W | N | A | S | W | S | N | K | S | | |
|---|---|---|---|---|---|---|---|---|---|---|---|---|---|---|---|---|
| Numèrotation | 610 | 611 | 612 | 613 | 614 | 615 | 616 | 617 | 618 | 619 | 620 | 621 | 622 | 623 | | |
| | L | E | Q | I | W | N | N | M | T | W | M | E | W | E | R | E |
| Numèrotation | 624 | 625 | 626 | 627 | 628 | 629 | 630 | 631 | 632 | 633 | 634 | 635 | 636 | 637 | 638 | 639 |
| | I | D | N | Y | T | S | L | I | Y | S | L | I | E | E | S | Q |
| Numèrotation | 640 | 641 | 642 | 643 | 644 | 645 | 646 | 647 | 648 | 649 | 650 | 651 | 652 | 653 | 654 | 655 |
| | N | Q | | | | | | | | | | | | | | |
| Numèrotation | 656 | 657 | | | | | | | | | | | | | | |

TABLE 3

SLEDIWDNMTWMQWEREIDNYTNTIYTLL
SLEQIWNNMTWMEWDREINNYTSLIHSLI
SLSDIWDNMTWMEWERETDNYTNLIYSLI
TLDQIWNNMTWMEWDREIDNYTHLIYTLI
SLNEIWQNMTWMEWEREIDNYTGLIYSLI
SLEQIWNHTTWMEWDREINNYTSLIHSLI
SLEEIWDNMTWMEWEREIDNYTSLIYTLI
SLDSIWNNMTWMEWEKEIENYTNTIYTLI
SFNEIWDNMTWMEWEREINNYTNLIYNLI
SLDDIWNNMTWMQWEKEISNYTGITYNLI
SLEQFWNNMTWMEWDREINNYTSLIHSLI
SLDDIWNNMTWMQWEREIDNYTSLIYSLL
SLDEIWQNMTWMEWEREIDNYTGLIYSLI
SLNEIWDNMTWMQWEREIDNYTHLIYTLI
SLNNIWNNMTWMQWERETDNYTGIIYNLL
SLDQIWNNMTWMEWEREIDNYTNLIYTLI
SLDKIWNNMTWMEWEREIDNYTSLIYTLL
SLDKIWGNMTWMEWEREIDNYTSLIYTLI
SMDQIWNNMTWMEWEREIDNYTSLIYNLI
SMNQIWDNLTWMEWEREIDNYTSITYSLI
SLNEIWDNMTWMKWEREIDNYTHITYSLI
SLNDIWQNMTWMEWEREIDNYTGLIYRLI
SLEETWNNMTWIEWEREIDNYTGVIYSLI
SQSDIWDKMTWLEWDKEVSNYTQVIYNLI

What is claimed is:

1. A peptide containing from 25 to 40 amino acids and comprising a sequence π corresponding to formula I:
WX₁X₂WX₃X₄EX₅X₆NYT
wherein:
X1 represents an amino acid selected from the group consisting of amino acids M, I, L and Q;
X₂ represents an amino acid selected from the group consisting of amino acids E, K, R, and Q;
X₃ represents an amino acid selected from the group consisting of amino acids D and E;
X₄ represents an amino acid selected from the group consisting of amino acids K, R, and Q;
X₅ represents an amino acid selected from the group consisting of amino acids V, I, and L; and
X₆ represents an amino acid selected from the group consisting of amino acids D, S, N, and E;
and wherein letters in formula I other than X represent amino acids according to conventional nomenclature.

2. A peptide according to claim 1, wherein the polypeptide corresponds to formula II:
(X₇)ₘ-π-(X₈)ₙ
wherein
π corresponds to the definition in claim 1;
X₇ represents a peptides containing at most 28 amino acids;
X₈ represents a peptides containing at most 28 amino acids;
or X₇ and X₈ together represent a divalent peptide which forms with π a cyclic peptide;
m is 0 or 1, n is 0 or 1; with the proviso that n and m cannot at the same time be equal to 0.

3. A peptide according to claim 2, wherein X7, X8 or both correspond to an amino acid sequence such that the peptide of formula II has a secondary structure corresponding to an α helix.

4. A peptide according to claim 2 or claim 3, wherein X7 corresponds to the formula:
(X₇″)_{m′}-X₇′-
wherein X7 represents amino acid T₆₃₂ of a gp41 protein from an HIV-1 strain or a fragment contained in a sequence corresponding to amino acids 610 to 632 of a gp41 protein from an HIV-1 strain, a consensus sequence corresponding thereto, or a sequence obtained by replacing in a fragment contained in a sequence corresponding to amino acids 610 to 632 of a gp41 protein from an HIV-1 strain or a consensus sequence corresponding thereto K with R, R with K, I with L, L with I or K and R with Q, wherein X7′ is linked to [symbol] through amino acid 632,
X7″ represents one amino acid or a fragment of a peptide containing at most 27 amino acids; and
m′ is 0 or 1.

5. A peptide according to claim 4, wherein the amino acids forming the fragment contained in a sequence corresponding to amino acids 610 to 632 of a gp41 protein from an HIV-1 strain are selected from corresponding amino acids in the sequences of Tables 1 and 2.

6. A peptide according to claims 2, 3 or 4 wherein X8 corresponds to the formula:
-X₈′-(X₈″)_{n′}
wherein X8′ represents N₆₄₅, S₆₄₅, G₆₄₅, H₆₄₅ or Q₆₄₅ of a gp41 protein from an HIV-1 strain or a fragment contained in a sequence corresponding to amino acids 657 to 657 of a gp41 protein from an HIV-1 strain, a consensus sequence corresponding thereto, or a sequence obtained by replacing in a fragment contained in a sequence corresponding to amino acids 645 to 657 of a gp41 protein from an HIV-1 strain or a consensus sequence corresponding thereto K with R, R with K, I with L, L with I or K and R with Q, wherein X8′ is linked to [symbol] through amino acid 645,
X8″ represents one amino acid or a fragment of a peptide containing at most 27 amino acids; and
n′ is 0 or 1.

7. A peptide according to claim 6, wherein the amino acids forming the fragment contained in a sequence corresponding to amino acids 645 to 657 of a gp41 protein from an HIV-1 strain are selected from corresponding amino acids in the sequences of Tables 1 and 2.

8. A therapeutic agent comprising one or more peptides according to claims 1-7 in combination with an excipient and/or a pharmaceutical carrier.

9. A method of treating a subject infected with HIV virus, wherein the method comprises administering to the subject a therapeutically effective amount of a therapeutic agent comprising one or more peptides of claims 1-7.
